(19) United States
(12) Patent Application Publication
(10) Pub. No.: US 2022/0309445 A1
(43) Pub. Date: Sep. 29, 2022

(54) PHARMACEUTICAL INVENTORY MANAGEMENT

(71) Applicant: Medimpact Healthcare Systems, Inc., San Diego, CA (US)

(72) Inventors: Greggory N. Gehrich, Rancho Santa Fe, CA (US); Gary C. Reiss, Newtown, PA (US)

(21) Appl. No.: 17/675,964

(22) Filed: Feb. 18, 2022

Related U.S. Application Data
(63) Continuation of application No. 13/224,683, filed on Sep. 2, 2011.
Application No. 13/224,683 is a continuation of application No. 13/011,608, filed on Jan. 21, 2011, now abandoned.
(60) Provisional application No. 61/275,719, filed on Jan. 22, 2010.

(51) Int. Cl. G06Q 10/08; G16H 20/13; G16H 10/60

(52) U.S. Cl. CPC G06Q 10/087; G16H 20/13; G16H 10/60

(57) ABSTRACT

Computer-implemented systems and methods for synchronizing network databases for an electronic inventory manager. The system includes a computer network and a remote database communicatively coupled to the computer network. The remote database is configured to store information related to whether a prescription drug is included in a select program at the time of processing a prescription drug claim. The system includes a client device configured to synchronize a quantity of the prescription drug claimed by the patient over the computer network and approve the claim under the select program. The system includes an electronic inventory system configured to determine whether the prescription drug claim is eligible to be fulfilled with the select program and synchronize the value of the inventory level at the electronic inventory system associated with the select program.

### CROSS REFERENCE TO RELATED APPLICATION

This application claims the benefit of and priority to and is a continuation of U.S. patent application Ser. No. 13/224,683, filed on Sep. 2, 2011, entitled “SYSTEMS AND METHODS FOR PROCESSING PRESCRIPTIONS,” which is a continuation of U.S. patent application Ser. No. 13/011,608, filed on Jan. 21, 2011, entitled “SYSTEMS AND METHODS FOR PROCESSING PRESCRIPTIONS,” which claims priority to U.S. Provisional Application No. 61/275,719, filed on Jan. 22, 2010, and titled “SYSTEMS AND METHODS FOR PROCESSING PRESCRIPTIONS,” the entirety of each of which is incorporated by reference herein.

### FIELD

The present technology relates to systems and methods for managing claims over a network. More particularly, the technology relates to computer architecture and operating methods that can allow for real-time synchronization of a local database with a network database having an electronic inventory manager.

### BACKGROUND

Computing systems can include a processor, a memory, a storage device, and input/output devices. The processor, the memory, the storage device, and the input/output devices can be interconnected via a system bus. The processor is capable of processing instructions for execution within the computing system. Such executed instructions can implement one or more components of, for example, a cloud platform. The computing system may include input/output devices that can provide input/output operations for a network device. For example, the input/output device can include Ethernet ports or other networking ports to communicate with one or more wired and/or wireless networks (e.g., a local area network (LAN), a wide area network (WAN), the Internet) or hardware or software implemented communications switches placed within the networked environment.

In conventional database systems, users access their data resources in one logical database. A user of such a conventional system typically retrieves data from and stores data on the system using the user's own systems. A user system might remotely access one of a plurality of server systems that might in turn access the database system. Data retrieval from the system might include the issuance of a query from the user system to the database system. The database system might process the request for information received in the query and send to the user system information relevant to the request. The rapid and efficient retrieval of accurate information is critical to fulfilling prescription medication in a computer network environment in which a plurality of computing systems access and retrieve data from a plurality of databases coupled in a communications network

Typically, a patient who receives a prescription from a medical professional goes to a pharmacy to have the prescription fulfilled. The pharmacist may then send the prescription information to a program processer such as a pharmacy benefit manager (PBM), a 340B processer, etc. for adjudication of a “claim” for fulfillment of the prescription. The program processer may manage health plans for various sponsors (e.g., employers, managed care organizations, labor unions, and state-funded pharmaceutical assistance programs). The program processer checks on the eligibility of the patient to receive the prescribed drug based on the coverage provided by the sponsor. For example, where a patient is insured and a sponsor covers a portion of the cost of the prescription, the program processer may determine how much the patient owes as a co-payment and the amount that will be charged to the sponsor for that particular drug. The program processer then informs the pharmacy that the claim has been approved and the pharmacy dispenses the drug to the patient. The patient may be required to provide a co-pay to the pharmacy. Further, the pharmacy may receive compensation such as a dispensing fee from the program processer. The program processer may charge this fee to the sponsor for payment.

The prescription medication fulfillment and payment processes can be very complicated, and in some cases, are fraught errors and inefficiencies that result in lost revenue for the sponsors and program processers. Also, in many cases, the errors can result in inconvenience to and overpayment by patients.

### SUMMARY

The systems, methods, and devices of described herein each may have several aspects, no single one of which is solely responsible for its desirable attributes. Without limiting the scope of this disclosure as expressed by the claims which follow, its more prominent features will now be discussed briefly. After considering this discussion, and particularly after reading the section entitled “Detailed Description” one will understand how the features of this technology provide advantages that include, without being limited thereto, dynamic determinations, including in real time, of how to process a claim for a prescription drug.

In accordance with certain embodiments, a computer-implemented inventory system is provided. The inventory system includes a first electronic inventory for maintaining prescription drugs eligible under a select program. The inventory system includes a second electronic inventory for maintaining prescription drugs not eligible under the select program coupled in a communications network, the first electronic inventory and the second electronic inventory being connected to one or more databases in a communications network. The inventory system also includes a network interface card configured to electronically receive information over the communications network from a pharmacy computer system, the information indicative of a prescription drug claim for a prescription drug associated with a patient of a pharmacy. The inventory system includes a processor configured to retrieve from a first database information about the prescription drug, the information comprising a plurality of values for the prescription drug retrieved automatically in real-time at the time of processing the prescription drug claim from the one or more databases over the communications network, at least a first one of the plurality of values being associated with the first inventory and at least a second one of the plurality of values being associated with the second inventory, wherein at least one of the one or more databases is implemented as a remote database from which the information is retrieved and stored in a list locally accessible by the inventory system. The processor is further configured to determine if the prescription drug claim is eligible to be fulfilled under the select program based at least in part upon the inclusion of the particular prescription drug in the select program according to the information retrieved from the first database over the communications network at the time of processing the prescription drug claim and a value of a first inventory level for the first inventory associated with the select program. The processor is further configured to approve the prescription drug claim under the select program, in response to determining dispensing the prescription drug from the first inventory is (1) more advantageous to a patient requesting the prescription drug and (2) a value of the inventory level for the prescription drug as associated with the first inventory is above a threshold value. The processor is further configured to transmit notification of the claim approval and reduce the value of the inventory level associated with the first inventory for the select program by an amount of the prescription drug claim, in response to transmitting the notification of the claim approval.

In another aspect, a non-transitory computer-readable storage medium storing instructions that, when executed by a processor, cause the processor to perform operations is provided. The operations include receiving, over a computer network, information indicative of a prescription drug claim for a prescription drug associated with a patient. The operations include determining, from a remote database communicatively coupled to the computer network, whether the prescription drug is included in a select program at the time of processing the prescription drug claim. The operations include determining, based on an electronic inventory system communicatively coupled to the remote database and in response to determining the prescription drug is included in the select program, whether the prescription drug claim is eligible to be fulfilled under the select program based at least in part upon a value of an inventory level associated with the select program at the time of processing the prescription drug claim, the inventory level managed by the electronic inventory system communicatively coupled to the remote database, and the inventory level associated with the select program being separate from an inventory level not associated with the select program. The operations include determining, based on the electronic inventory system communicatively coupled to the remote database, the value of the inventory level associated with the select program is above a threshold value. The operations include approving the claim under the select program in response to determining the value of the inventory level associated with the select program is above the threshold value. The operations include providing, over the computer network, an instruction to the electronic inventory system to synchronize the value of the inventory level at the remote database associated with the select program by a quantity of the prescription drug claim claimed by the patient. The select program may be a discount program.

In yet another aspect, an electronic inventory system communicatively coupled to one or more databases and client devices in an electronic network for updating inventory data in at least one of the databases, the electronic inventory system comprising at least one processor configured to perform operations is provided. The operations include determining whether a prescription drug included in a select program, according to data stored in a database remotely coupled to the electronic inventory system, is eligible to be filled under the select program based at least in part on a first inventory level value associated with a first inventory maintained separately for the select program at the time of processing a prescription drug claim, the first inventory managed by the electronic inventory system communicatively coupled to the remote database, and the first inventory being separate from a second inventory other than the first inventory, the second inventory being associated with prescription drugs not qualified under the select program. The operations include determining, based on the electronic inventory system, the first inventory level value associated with the select program is above a threshold value. The operations include approving the prescription drug claim under the select program. The operations include updating, based on a quantity of the prescription drug approved, the first inventory level value.

One aspect of the disclosure is a computer-implemented method of adjudicating prescription drug claims. The method comprises receiving information indicative of a prescription drug claim for a prescription drug from a pharmacy. The method further comprises automatically retrieving pricing information for the prescription drug, the pricing information comprising a plurality of prices for the prescription drug, each of the plurality of prices being associated with a different rate. The method further comprises determining if the prescription drug claim is eligible to be fulfilled under a discount program. The method further comprises calculating a first actual cost for the prescription drug under a standard network rate. The method further comprises calculating a second actual cost for the prescription drug under a discount program rate of the discount program. The method further comprises determining the lesser of the first actual cost and the second actual cost. The method further comprises approving the claim under the lesser of the first actual cost and the second actual cost.

Another aspect of the disclosure is a system for adjudicating prescription drug claims, the system comprising a processor. The system further comprises circuitry coupled to the processor. The circuitry and the processor are cooperatively configured to receive information indicative of a prescription drug claim for a prescription drug from a pharmacy. The circuitry and the processor are further cooperatively configured to automatically retrieve pricing information for the prescription drug, the pricing information comprising a plurality of prices for the prescription drug, each of the plurality of prices being associated with a different rate. The circuitry and the processor are further cooperatively configured to determine if the prescription drug claim is eligible to be fulfilled under a discount program. The circuitry and the processor are further cooperatively configured to calculate a first actual cost for the prescription drug under a standard network rate. The circuitry and the processor are further cooperatively configured to calculate a second actual cost for the prescription drug under a discount program rate of the discount program. The circuitry and the processor are further cooperatively configured to determine the lesser of the first actual cost and the second actual cost. The circuitry and the processor are further cooperatively configured to approve the claim under the lesser of the first actual cost and the second actual cost.

Yet another aspect of this disclosure is a computer-readable medium having computer executable instructions stored thereon. The stored instructions, when executed by a processor, cause the processor to perform a method of adjudicating prescription drug claims. The method comprises receiving information indicative of a prescription drug claim for a prescription drug from a pharmacy. The method further comprises automatically retrieving pricing information for the prescription drug, the pricing information comprising a plurality of prices for the prescription drug, each of the plurality of prices being associated with a different rate. The method further comprises determining if the prescription drug claim is eligible to be fulfilled under a discount program. The method further comprises calculating a first actual cost for the prescription drug under a standard network rate. The method further comprises calculating a second actual cost for the prescription drug under a discount program rate of the discount program. The method further comprises determining the lesser of the first actual cost and the second actual cost. The method further comprises approving the claim under the lesser of the first actual cost and the second actual cost.

A further aspect of this disclosure is a system for adjudicating prescription drug claims, the system comprising means for receiving information indicative of a prescription drug claim for a prescription drug from a pharmacy. The system further comprises means for automatically retrieving pricing information for the prescription drug, the pricing information comprising a plurality of prices for the prescription drug, each of the plurality of prices being associated with a different rate. The system further comprises means for determining if the prescription drug claim is eligible to be fulfilled under a discount program. The system further comprises means for calculating a first actual cost for the prescription drug under a standard network rate. The system further comprises means for calculating a second actual cost for the prescription drug under a discount program rate of the discount program. The system further comprises means for determining the lesser of the first actual cost and the second actual cost. The system further comprises means for approving the claim under the lesser of the first actual cost and the second actual cost.

For the embodiments described above and elsewhere herein, the information indicative of a prescription drug claim may include, for example, an identifier of the pharmacy, an identifier of a source of a prescription for the prescription drug, and an identifier of the prescription drug. Further, the discount program may include, for example, a 3409 program. Additionally, determining if the prescription drug is eligible to be fulfilled under the government program may include, for example, determining if the pharmacy is a 3409 eligible pharmacy. Determining if the prescription drug is eligible to be fulfilled under the government program may further include, for example, determining if the source of the prescription is a 3409 eligible source, such as a 3409 authorized physician. Further yet, determining if the prescription drug is eligible to be fulfilled under the government program may include, for example, determining if the pharmacy is associated with the source.

In addition, for the embodiment described above and elsewhere herein, determining if the prescription drug is eligible to be fulfilled under the government program may include determining if a patient associated with the prescription drug claim is a 3409 eligible patient. Further, determining if the prescription drug is eligible to be fulfilled under the government program may include determining if the prescription drug is a 340B eligible drug.

Yet further, automatically retrieving pricing information for the prescription drug may include retrieving pricing information from a price data bank at a regular time interval, wherein the interval may be, for example, daily, twice daily, weekly, or bi-weekly. The discount program may include a group purchasing organization (GPO) program.

The embodiments described above and elsewhere herein may further include automatically maintaining an inventory of drugs. The embodiments may further include transmitting the approval to the pharmacy for fulfillment of the claim.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, not to be considered limiting of its scope, the disclosure will be described with additional specificity and detail through use of the accompanying drawings.

FIG. 1 depicts a non-limiting example of a system for selectively adjudicating prescription claims under either a standard rate or a government discount program.

FIG. 2 illustrates a functional block diagram of one non-limiting example of a computer of FIG. 1.

FIG. 3 depicts a non-limiting example of a process for adjudication a prescription drug claim.

### DETAILED DESCRIPTION OF EXAMPLE EMBODIMENTS

The following detailed description is directed to certain specific embodiments. However, the teachings herein can be applied in a multitude of different ways, including for example, as defined and covered by the claims. It should be apparent that the aspects herein may be embodied in a wide variety of forms and that any specific structure, function, or both being disclosed herein is merely representative. Based on the teachings herein one skilled in the art should appreciate that an aspect disclosed herein may be implemented independently of any other aspects and that two or more of these aspects may be combined in various ways. For example, a system or apparatus may be implemented or a method may be practiced using any number of the aspects set forth herein. In addition, such a system or apparatus may be implemented or such a method may be practiced using other structure, functionality, or structure and functionality in addition to or other than one or more of the aspects set forth herein. In this description, reference is made to the drawings wherein like parts are designated with like numerals throughout.

Various embodiments of systems and methods are described herein for processing prescription drug claims. In the embodiments described herein, the systems and methods may allow prescription drug claims to be processed in a more efficient and cost effective manner. For example, the systems and methods described herein may advantageously allow pharmacies to automatically determine which program to adjudicate a drug claim among several programs at a point of acquisition. This can lead to reduced costs for patients. This can also lead to time saved at the pharmacy, which can reduce the cost of running the pharmacy.

Manufacturers of prescription drugs typically set standard rates for the drugs that they sell. In some cases, these rates may be negotiated by the program processor on behalf of sponsors who provide healthcare coverage to a group of individuals. Unfortunately, these prices may be prohibitively high for many individuals. Further, in the case of people who have no sponsor, they may be forced to pay the full price for drugs out of their own pocket.

In order to alleviate the problem of high drug costs, various discount drug programs have been introduced. Non-limiting examples of such programs include the 340B program, group purchasing organizations (GPOs), and the like. The discount drug programs allow eligible individuals/groups to receive drugs at reduced rates. For example, drug manufacturers may agree to provide certain drugs at a discounted rate under certain programs. The programs may have certain criteria for determining whether a given individual is covered by the program or not. Accordingly, if an individual is covered by such a program, the drug manufacturer may provide the drug at a discounted rate that may be less than the standard rate for the drug.

The various reimbursement mechanisms can result in great complexity for the prescription drug fulfillment and payment processes. For example, those complexities often result in a drug being administered under an incorrect mechanism, which may result in added cost for the patient, the patient's sponsor and the program processer. For example, the cost of a drug administered under one mechanism may be lower than the cost of the same drug administered under a different mechanism. Without a method to determine which is the lower cost mechanism, it is possible the drug may be administered under the higher cost mechanism. This can potentially lead to higher costs for the patients in the form of a higher co-pay, as well as a higher cost for sponsors, program processor, and pharmacies as they may also bear the portions of expense of the higher cost mechanism. Further, since drug administration occurs frequently around the country at numerous pharmacies, these costs can add up to significant sums of money.

Some of the embodiments described herein relate to methods, systems and devices that can be used to determine whether to adjudicate a prescription medication claim under a standard rate or under some discount drug program to advantageously acquire the drug at the lowest cost. The cost reductions from always choosing the lowest cost method of adjudication can be passed on to any combination of program processers, pharmacies, patients, sponsors, and the like, which can advantageously lower costs incurred in providing health care to all individuals. Some embodiments described herein may, for example, automatically and in real-time determine whether an individual is eligible for discount drug pricing under a discount drug program, determine whether the cost would be less to adjudicate the claim under one or more discount drug program or a standard rate, and process the claim under the lowest cost program. By automatically determining eligibility and/or cost dynamically, including in real-time or at regular intervals (e.g., daily, weekly, biweekly), as opposed static systems and methods that make manual determinations on an infrequent basis or even automated determinations on an infrequent basis (e.g., quarterly), many potential problems can be averted. For example, proper co-pays can be calculated for patients at the point of acquisition. If the eligibility and cost is not determined at the point of acquisition, there may be no practical way of retroactively revising the co-pay of a patient, allowing them to share in the lower drug pricing, after the patient leaves the pharmacy. Further, potential regulatory problems can be averted. For example, if a patient is allowed to receive a drug at a discounted rate when the patient is not actually eligible for the discounted rate, there can be serious legal consequences for not following the regulatory guidelines. For illustrative purposes, the below described embodiments are discussed with respect to the government 340B program. However, with the benefit of the disclosure found herein, one of ordinary skill in the art will recognize that other embodiments may similarly use alternative/additional discount drug programs. Further, one of ordinary skill in the art will recognize that other embodiments may similarly evaluate and determine the lowest cost as between standard rates for brand name drugs, discount rates for brand name drugs, standard rates for generic drugs, and discount rates for generic drugs.

The government 340B program is a drug discount program codified as Section 340B of the Public Health Service Act. Section 340B limits the cost of covered outpatient drugs to certain federal grantees, federally-qualified health center look-alikes and qualified disproportionate share hospitals (collectively referred to herein as “clinics”). Significant savings on pharmaceuticals may be seen by those entities that participate in this program. Each of these clinics has associated with them a particular pharmacy that is authorized to dispense drugs at the 340B discount to patients that obtain their prescription from the associated clinic. Accordingly, currently a patient's eligibility for the 340B program is based on whether the pharmacy that the patient goes to fulfill the prescription is a 340B eligible pharmacy, and whether the clinic from where the prescription was obtained is the 340B eligible clinic associated with that particular 340B pharmacy.

The government 340B program further requires that the drugs dispensed by a pharmacy at 340B pricing be from a separate 340B inventory. For example, the clinic can request the drugs from a wholesaler. The wholesaler then ships the drugs to the pharmacy and bills the clinic. The program processor or another party may keep track of the drugs on behalf of the clinic, so as to only sell the drugs that are acquired under the 340B program at the 340B discount. In one embodiment, the pharmacy may physically divide or segregate the 340B inventory from non-340B inventory (the standard inventory), and only gives the physical drugs acquired under the 340B program at the 340B discount. The standard inventory is used to fulfill non-340B prescription claims.

In another embodiment, the pharmacy may keep track of the drugs acquired under the 340B program by using a virtual inventory system. The virtual inventory system may keep track of the amount of drugs acquired under the 340B program as well as the drugs not acquired under the 340B program. When a drug is given to a patient under the 340B program, the amount given of that drug is deducted from the 340B inventory. When a drug is given to a patient based on a standard rate, the amount given of that drug is deducted from the pharmacy's own, non-340B inventory. The drugs are not physically separated, but the amount that can be dispensed under the 340B program is tracked to ensure that only the amount acquired under 340B is dispensed under 340B. In some embodiments, the virtual inventory may be managed by a virtual inventory manager. The virtual inventory manager may be, for example, a third party that maintains and updates one or more databases containing the inventory of one or more pharmacies.

The following methods and systems automate the process of adjudicating prescription claims based on the lower of cost of a standard network rate and a government discount program. The automated process advantageously allows claims to be immediately adjudicated at the lowest cost. The automated process saves money by determining the lowest cost solution to adjudicating a claim. Further, the automatic process saves time by eliminating the need for post-adjudication adjustments requiring claims adjudicated under one process to be reversed and re-adjudicated by a second process. In one embodiment a processer can use a series of pre-defined edits to calculate the low net cost for a claim. The basis for this calculation may be a pricing list obtained for each national drug code (NDC) from a price databank. Further, the calculation can utilize the NDC pricing for a discount program, such as 340B. The calculations may be made and prices compared at the time of an acquisition of the drug based on the claim.

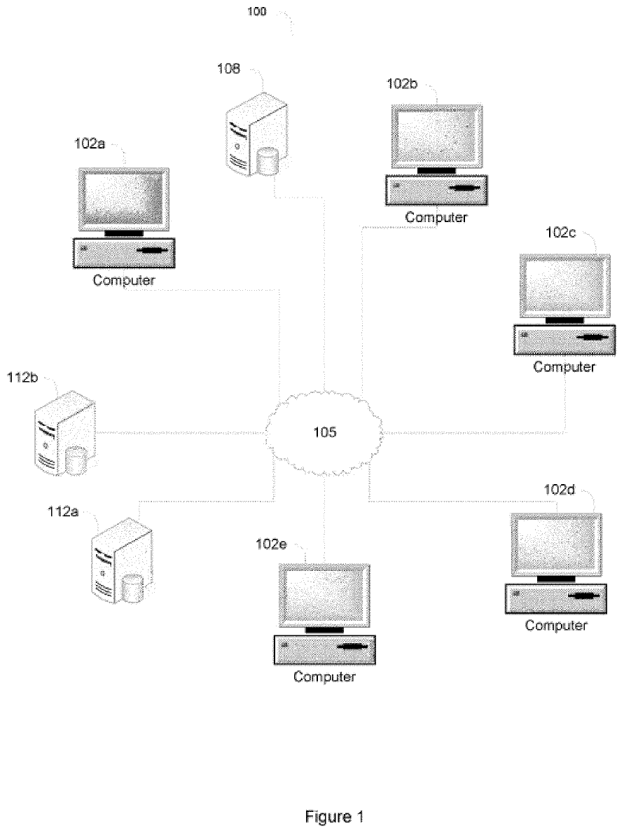

FIG. 1 depicts one example of a system for selectively adjudicating prescription claims under either a standard rate or a discount program. The system 100 is configured to support adjudication of claims. The system 100 includes one or more computers 102a-102e, which can be, for example, any workstation or computing device capable of processing, sending, and receiving information. Each of the computers 102a-102e can be connected, by any suitable communications technology (e.g., an internet protocol), to a network 105 (e.g., the Internet). Accordingly, the computers 102a-102e may transmit and receive information (e.g., claims, approvals, inventory updates, invoices, orders, etc.) between each other via the network 105.

Each of the computers 102a-102e may correspond to a computer at a different location, for example. For example, computer 102a may comprise a computer located at a pharmacy (“pharmacy computer 102a”). Further, computer 102b may comprise, for example, a computer located at a program processer (“program processer computer 102b”). Computer 102c may comprise, for example, a computer located at a virtual inventory manager (“inventory computer 102c”). Computer 102d may comprise, for example, a computer located at a wholesaler (“wholesaler computer 102d”). Computer 102e may comprise, for example, a computer located at a clinic (“clinic computer 102e”).

The pharmacy computer 102a may be configured to receive input from a pharmacist. For example, the computer 102a may include an input device. Non-limiting examples of input devices are described elsewhere herein. The pharmacist may enter information related to a prescription received from a patient into the computer 102a via the input device. The information may include one or more of, for example, an identifier of a drug (e.g., a national drug code (NDC), a prescription control number (PCN), etc.), the name of the patient, the clinic or doctor that wrote the prescription, health insurance information of the patient, etc. The pharmacy computer 102a may then transmit a prescription claim for adjudication via an appropriate interface (e.g., a network interface card) to the program processer computer 102b via the network 105. The prescription claim may include at least some of the information input by the pharmacist.

The program processer computer 102b may use the information received from the pharmacy computer 102a to determine how to process the claim. For example, the program processer computer 102b may determine whether the claim is eligible to be processed under a discount program. For example, where the discount program is the 340B program, the program processer computer 102b may determine whether the claim for a prescription was obtained by a patient at a 340B eligible clinic, for example according to 340B rules. If the program processer computer 102b determines the prescription was obtained from a 340B clinic, the program processer computer 102b may further determine whether the prescription was sent from the 340B eligible pharmacy that is associated with the particular 340B clinic from which the prescription was obtained. In one embodiment, the program processer computer 102b may base these determinations by matching information sent with the claim by the pharmacy 102a with information contained in a database 108 accessible by the program processer computer 102b. For example, the database 108 may contain information regarding which clinics are 340B eligible and which pharmacies are associated with each of those clinics. Further, the database 108 may contain information mapping physicians to clinics and/or pharmacies associated with those clinics. The information sent with the claim may include the name of the prescribing physician. Therefore the program processer computer 102b may map the prescribing physician with a clinic and then determine if that clinic is a 340B clinic associated with the pharmacy.

The database 108 may be, for example: located remotely from the program processer computer 102b and accessed via the network 105, located on another network such as a local network to which the program processer computer 102b is connected, and/or stored in a memory of the program processer computer 102b. The database 108 may further be updated manually or automatically as the list of 340B eligible clinics and associated pharmacies changes.

If the program processer computer 102b determines that the prescription of a claim was obtained from a 340B clinic, brought for fulfillment at the 340B pharmacy associated with that clinic, and the prescription drug of the claim is 340B eligible, the claim is eligible for 340B processing. Otherwise the claim is not eligible for 340B processing and is processed under the standard network rate. If the claim is eligible for 340B processing, the program processer computer 102b determines whether it would cost less to adjudicate the claim under the 340B program or if it would cost less to adjudicate the claim under a standard network rate. The program processer computer 102b may make the determination by looking up or by calculating a cost for adjudicating the claim under the standard network rate and calculating a cost for adjudicating the claim under the 340B program (or any other discount program).

In some embodiments, the program processer computer 102b may determine a drug of a claim is 340B eligible by checking/updating a list of eligible drugs stored in a database or list. The program processer computer 102b may retrieve information regarding eligible drugs from a server, which may comprise, for example, a database and/or computer connected to the network 105. In one embodiment the list of eligible drugs may be retrieved at regular intervals (e.g., daily, weekly, or biweekly) and a drug list stored on the program processer computer 102b may be updated at those intervals. In another embodiment, the list of eligible drugs may be retrieved in real-time, for example. By performing the retrieval dynamically, for example, in real-time or at regular intervals (e.g., daily, weekly, biweekly, etc.), new drugs that may be available may be properly included when determining the most cost effective method to process a claim. Since new drugs may become available under a particular program at any time, static methods that update less frequently may not include these new drugs and therefore may erroneously use a higher cost method of processing the claim.

In some embodiments, the program processer computer 102b may calculate the standard network rate adjudication cost as follows. The program processer computer 102b may retrieve information regarding standard network rates from a price data bank 112a, which may comprise, for example, a database and/or computer connected to the network 105. The price data bank 112a may contain, for example, information as to the standard price of the drug. For example, the price of a drug under the standard rate may be an average wholesale price of the drug (AWP) minus a percentage discount (e.g., 15%) plus a dispensing fee charged by the pharmacy. The standard network rates may be negotiated by the program processer. In one embodiment the prices may be retrieved at regular intervals (e.g., daily, weekly, biweekly) and a price list stored on the program processer computer 102b may be updated at those intervals. In another embodiment, the prices may be retrieved in real-time, for example. By performing the retrieval dynamically in real-time, new prices may be properly included when determining the most cost effective method to process a claim. Since prices may be updated under a particular program at any time, static methods may not include these new prices and therefore may erroneously use a higher cost method of processing the claim.

In yet another embodiment, the program processer can maintain a price list on the program processer computer 102b itself based on its own negotiated prices, for example. The program processer computer 102b may determine, for example, based on the retrieved information, the cost for adjudicating the claim for a prescription for a particular drug of a particular dosage.

The program processer computer 102b may also calculate a discount program adjudication cost (or any other non-standard rate adjudication cost), for example, the 340B program adjudication cost, as follows. The program processer computer 102b may retrieve information regarding 340B rates from a price data bank 112b (which may be the same or different than price data bank 112a), which may comprise, for example, a database and/or computer connected to the network 105. For example, the 340B rates may be obtained from a computer or price data bank 112b operated by the wholesaler. The 340B rates may be the rates of the wholesaler as they are negotiated with the clinic. The price data bank 112b may contain, for example, information as to the 340B price of the drug. For example, the price of a drug under the 340B program may be a dispensing fee charged by the pharmacy. The 340B rates may be set according to the government 340B program. In one embodiment the prices may be retrieved at regular intervals (e.g., daily, weekly, biweekly) and a price list stored on the program processer computer 102b may be updated at those intervals. In another embodiment, the prices may be retrieved in real-time. In yet another embodiment, the program processer may maintain a price list on the program processer computer 102b. The program processer computer 102b may determine, for example, based on the retrieved information, the cost for adjudicating the claim for a prescription for a particular drug of a particular dosage.

After calculating the price for adjudication of the drug under the standard network rate and the discount or non-standard rate, for example the 340B rate, the program processer computer 102b may determine whether to dispense the drug under the standard rate plan or under the 340B plan. For example, the program processer computer 102b can base the determination on which process would cost less. If the 340B rate is less, the program processer computer 102b starts adjudication of the claim under the 340B program. If the standard network rate is less, the program processer computer 102b starts adjudication of the claim under the standard network adjudication process. The program processer computer 102b sends back to the pharmacy computer 102a an approval for the claim. The approval for the claim may include, for example, whether the claim is to be processed under the standard process or under the 340B process.

In some aspects, for a claim approved under a discount program such as the 340B program, the program processer computer 102b may generate a bill or invoice to be sent (manually or electronically) to an acquiring entity that covers the cost of the drug and administrative fees. The acquiring entity then sends funds to the program processer (manually or electronically) to cover the cost of the drugs dispensed under the 340B program and administrative fees.

When the claim is approved under the 340B program, the program processer computer 102b or the pharmacy computer 102a further may report the approval to the inventory computer 102c. In one embodiment, the program processer computer 102b can transmit the report to the inventory computer 102c in real-time. In another embodiment, the program processer computer 102b stores one or more reports and transmits the one or more of reports to the inventory computer 102c at a regular interval (e.g., daily, weekly, biweekly). For example, the program processer computer 102b may post one or more reports to a file transfer protocol (FTP) server for the inventory computer 102c to retrieve.

The inventory computer 102c can track the amount of 340B inventory the pharmacy has for one or more drugs. Upon receiving the approval report, the inventory computer 102c deducts the dispensed amount of the drug from the 340B inventory, for example, from the virtual inventory. The inventory computer 102c is further configured to automatically request additional drugs if the 340B inventory of any particular drug is running low (e.g., the amount of the drug is below a threshold level). The inventory computer 102c may transmit the request for additional drugs to the wholesaler computer 102d via the network 105.

The wholesaler computer 102d may receive a request for additional drugs from the inventory computer 102c and/or the pharmacy 102a. The request may be for drugs to be acquired under a discount program, for example, such as the 340B program. The wholesaler computer 102d may then transmit a bill or invoice for the drugs to be acquired under the 340B program and send the bill to the clinic computer 102e. The wholesaler computer 102d may further automatically generate a shipment request for the drug to be sent to the pharmacy. The clinic may further receive funds from the program processer that the program processer received from an acquiring entity to pay for the drugs to be acquired under the 340B program. If it is the case that the clinic is the acquiring entity, the program processer computer 102a may adjust an invoice of the clinic to reflect the payment for the 340B program drugs.

It should be appreciated by one of ordinary skill in the art in view of the instant disclosure that one or more of the functions described above with respect to computers 102a-102e may be performed on other combinations of computers 102a-102e or additional computers. For example, one or more of the computers 102a-102e or the functions described in connection with those computers, may be combined on fewer computers or on, more different computers, or on one or more additional computers. Further, one or more of computers 102a-102e, database 108, and price bank 112a, 112b may be combined in one location and controlled by a single entity.

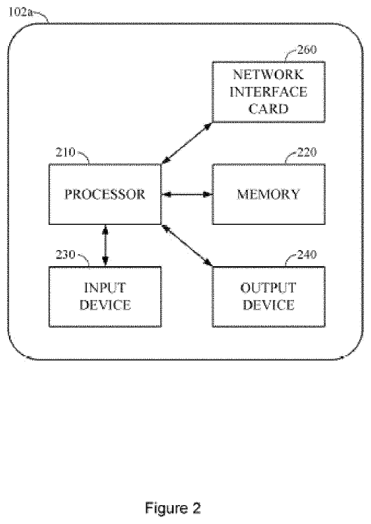

FIG. 2 illustrates a functional block diagram of one example of a computer of FIG. 1. The computer 102a includes a processor 210 in data communication with a memory 220, an input device 230, and an output device 240. The processor is further in data communication with a network interface card 260. Although described separately, it is to be appreciated that functional blocks described with respect to the computer 102a need not be separate structural elements. For example, the processor 210 and memory 220 may be embodied in a single chip.

The processor 210 can be a general purpose processor, a digital signal processor (DSP), an application specific integrated circuit (ASIC), a field programmable gate array (FPGA) or other programmable logic device, discrete gate or transistor logic, discrete hardware components, or any suitable combination thereof designed to perform the functions described herein. A processor may also be implemented as a combination of computing devices, e.g., a combination of a DSP and a microprocessor, a plurality of microprocessors, one or more microprocessors in conjunction with a DSP core, or any other such configuration.

The processor 210 can be coupled, via one or more buses, to read information from or write information to memory 220. The processor may additionally, or in the alternative, contain memory, such as processor registers. The memory 220 can include processor cache, including a multi-level hierarchical cache in which different levels have different capacities and access speeds. The memory 220 can also include random access memory (RAM), other volatile storage devices, or non-volatile storage devices. The storage can include hard drives, optical discs, such as compact discs (CDs) or digital video discs (DVDs), flash memory, floppy discs, magnetic tape, and Zip drives.

The processor 210 also may be coupled to an input device 230 and an output device 240 for, respectively, receiving input from and providing output to, a user of the computer 102a. Suitable input devices include, but are not limited to, a keyboard, buttons, keys, switches, a pointing device, a mouse, a joystick, a remote control, an infrared detector, a bar code reader, a scanner, a video camera (possibly coupled with video processing software to, e.g., detect hand gestures or facial gestures), a motion detector, or a microphone (possibly coupled to audio processing software to, e.g., detect voice commands). Suitable output devices include, but are not limited to, visual output devices, including displays and printers, audio output devices, including speakers, headphones, earphones, and alarms, and haptic output devices.

The processor 210 further may be coupled to a network interface card 260. The network interface card 260 prepares data generated by the processor 210 for transmission via a network according to one or more data transmission protocols. The network interface card 260 also decodes data received via a network according to one or more data transmission protocols. The network interface card 260 can include a transmitter, receiver, or both. In other embodiments, the transmitter and receiver can be two separate components. The network interface card 260, can be embodied as a general purpose processor, a digital signal processor (DSP), an application specific integrated circuit (ASIC), a field programmable gate array (FPGA) or other programmable logic device, discrete gate or transistor logic, discrete hardware components, or any suitable combination thereof designed to perform the functions described herein.

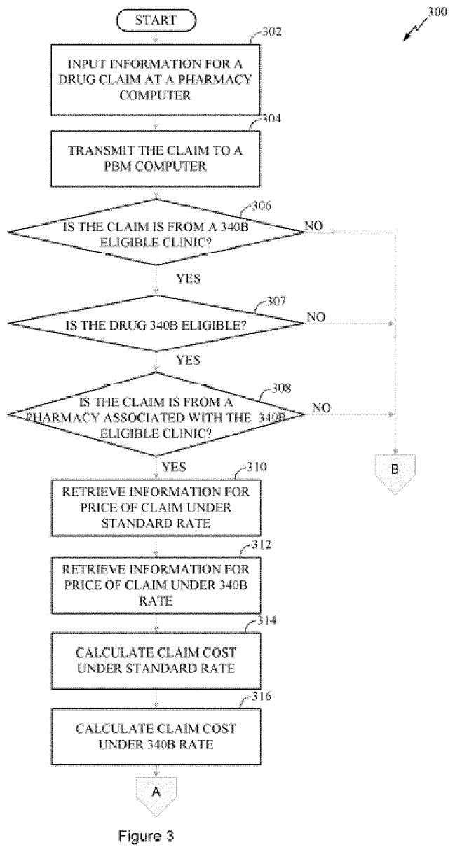

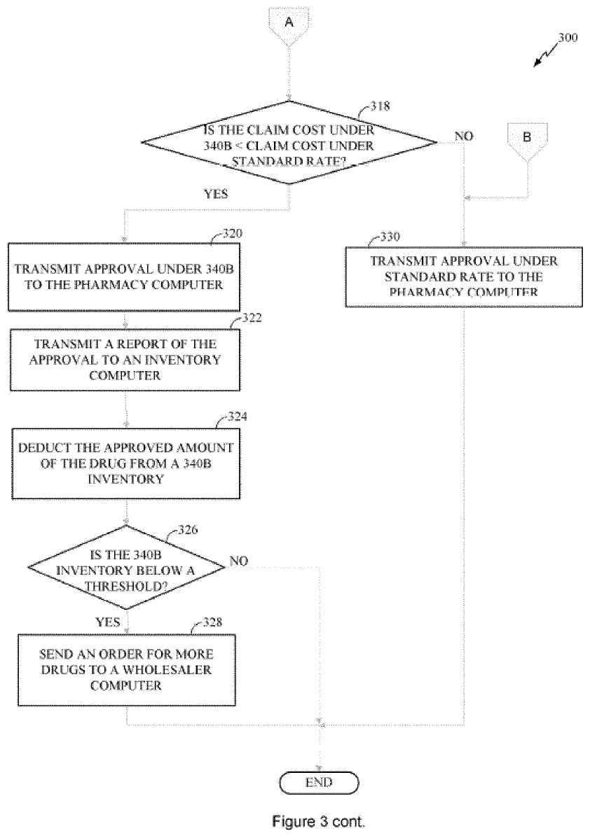

FIG. 3 depicts one example of a process for adjudication a prescription drug claim. The process 300 starts at a step 302 where information for a prescription drug claim is input into a pharmacy computer, for example, a computer such as computer 102a described elsewhere herein. Continuing at a step 304, the pharmacy computer (e.g., 102a) transmits the claim information to a program processer, for example, a computer such as computer 102b described elsewhere herein. Further, at a step 306, the program processer computer (e.g., 102b) determines whether the claim is for a prescription written from a 340B eligible clinic. If at the step 306, the program processer computer 102b determines the claim is not for a prescription written from a 340B eligible clinic, the process 300 continues to a step 330. If at the step 306, the program processer computer determines the claim is for a prescription written from a 340B eligible clinic, the process 300 continues to a step 307.

At the step 307, the program processer computer 102b determines whether the prescription drug of the claim is a drug that is eligible for the 340B program. If the program processer computer determines the prescription drug is not eligible for the 340B program, the process 300 continues to step 330. If the program processer computer determines the prescription drug is eligible for the 340B program, the process 300 continues to step 308.

At the step 308, the program processer 102b computer determines whether the claim was sent by a pharmacy associated with the 340B eligible clinic determined in step 306. If the program processer computer 102b determines the claim was not sent by a pharmacy associated with the 340B eligible clinic, the process 300 continues to step 330. If the program processer computer determines the claim was sent by a pharmacy associated with the 340B eligible clinic, the process 300 continues to step 310. In the embodiment of FIG. 3, this ends the eligibility checking.

Further, the financial analysis begins at a step 310. At the step 310, the program processer computer retrieves information from a price data bank, for example a data bank such as price data bank 112a described herein, information on the pricing of a drug or drug under a standard network rate. Continuing at a step 312, the program processer computer 102b retrieves information regarding the pricing of a drug or drugs under the 340B program from a price data bank 112b. Further, at a step 314 the program processer computer 102b calculates the cost of adjudicating the claim using a standard network rate based on the drug of the claim and the information retrieved form the price data bank (e.g., data bank 112a). At another step 316 the program processer computer (e.g., computer 102b) calculates the cost of adjudicating the claim under the 340B program based on the drug of the claim and the information retrieved form the price data bank.

Continuing at a step 318, the program processer computer 102b determines whether the cost of adjudicating the claim under the 340B program calculated at step 316 is less than the cost of adjudicating the claim using a standard network rate calculated at step 314. If the program processer computer determines the cost of adjudicating the claim under the 340B program is not less than the cost of adjudicating the claim using a standard network rate, the process 300 continues to step 330. If the program processer computer determines the cost of adjudicating the claim under the 340B program is less than the cost of adjudicating the claim using a standard network rate, the process 300 continues to step 320.

Further at step 320, the program processer computer transmits approval for the claim under the 340B program to the pharmacy computer. Next at the step 322, the program processer computer transmits a report of the approval to the inventory computer, for example an inventor computer such as computer 102c described herein. The inventory computer (e.g., computer 102c), at a further step 324, deducts the amount of the drug approved in the claim from an inventory for the pharmacy. Next, at a step 326, the inventory computer determines whether a 340B inventory of the pharmacy is below a threshold level. If the inventory computer determines the 340B inventory of the pharmacy is not below a threshold level, the process 300 ends. If the inventory computer determines the 340B inventory of the pharmacy is below a threshold level, the process continues to at step 328. At the step 328, the inventory computer sends a request for more drugs to a wholesaler computer to replenish the depleted inventory. The process 300 then ends.

At a step 330, the program processer computer transmits approval for the claim under a standard network rate to the pharmacy computer. The pharmacy then handles the dispensing of the drug and manages its own inventory. Accordingly, the process 300 then ends.

One or ordinary skill in the art should recognize that various steps may by added or omitted from the process 300. Further, the various steps of the process 300 may be performed in a different order than described above. Although FIG. 3 depicts a process associated with adjudicating a 340B or standard rate drug dispensing process, it should be understood that the process and steps can be used with other non-340B discount programs or any other drug dispensing programs.

The technology is operational with numerous other general purpose or special purpose computing system environments or configurations. Examples of well known computing systems, environments, and/or configurations that may be suitable for use with the technology disclosed herein include, but are not limited to, personal computers, server computers, hand-held or laptop devices, multiprocessor systems, microprocessor-based systems, programmable electronics, network PCs, minicomputers, mainframe computers, distributed computing environments that include any of the above systems or devices, and the like.

As used herein, instructions refer to computer-implemented steps for processing information in the system. Instructions can be implemented in software, firmware or hardware and include any type of programmed step undertaken by components of the system.

A Local Area Network (LAN) or Wide Area Network (WAN) may be a corporate computing network, including access to the Internet, to which computers and computing devices comprising the system are connected. In one embodiment, the LAN conforms to the Transmission Control Protocol/Internet Protocol (TCP/IP) industry standard.

As used herein, media refers to images, sounds, video or any other multimedia type data that is entered into the system.

A microprocessor may be any conventional general purpose single- or multi-chip microprocessor such as a Pentium® processor, a Pentium® Pro processor, a 8051 processor, a MIPS® processor, a Power PC® processor, or an Alpha® processor. In addition, the microprocessor may be any conventional special purpose microprocessor such as a digital signal processor or a graphics processor. The microprocessor typically has conventional address lines, conventional data lines, and one or more conventional control lines.

The system is comprised of various modules as discussed in detail. As can be appreciated by one of ordinary skill in the art, each of the modules comprises various subroutines, procedures, definitional statements and macros. Each of the modules are typically separately compiled and linked into a single executable program. Therefore, the description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

The system may be used in connection with various operating systems such as Linux®, UNIX® or Microsoft Windows®.

The system may be written in any conventional programming language such as C, C++, BASIC, Pascal, or Java, and ran under a conventional operating system. C, C++, BASIC, Pascal, Java, and FORTRAN are industry standard programming languages for which many commercial compilers can be used to create executable code. The system may also be written using interpreted languages such as Perl, Python or Ruby.

A web browser comprising a web browser user interface may be used to display information (such as textual and graphical information) to a user. The web browser may comprise any type of visual display capable of displaying information received via a network. Examples of web browsers include Microsoft's Internet Explorer browser, Netscape's Navigator browser, Mozilla's Firefox browser, PalmSource's Web Browser, Apple's Safari, or any other browsing or other application software capable of communicating with a network.

Those of skill will further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In one or more example embodiments, the functions and methods described may be implemented in hardware, software, or firmware executed on a processor, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the above description has pointed out novel features of the technology as applied to various embodiments, the skilled person will understand that various omissions, substitutions, and changes in the form and details of the device or process illustrated may be made without departing from the scope of the instant technology. Therefore, the scope of the technology is defined by the appended claims rather than by the foregoing description. All variations coming within the meaning and range of equivalency of the claims are embraced within their scope.

What is claimed is:

1. A computer-implemented inventory system comprising:
a first electronic inventory for maintaining prescription drugs eligible under a select program;
a second electronic inventory for maintaining prescription drugs not eligible under the discount program coupled in a communications network, the first electronic inventory and the second electronic inventory being connected to one or more databases in a communications network;
a network interface card configured to electronically receive information over the communications network from a pharmacy computer system, the information indicative of a prescription drug claim for a prescription drug associated with a patient of a pharmacy; and
a processor configured to:
retrieve from a first database information about the prescription drug, the information comprising a plurality of values for the prescription drug retrieved automatically in real-time at the time of processing the prescription drug claim from the one or more databases over the communications network, at least a first one of the plurality of values being associated with the first inventory and at least a second one of the plurality of values being associated with the second inventory, wherein at least one of the one or more databases is implemented as a remote database from which the information is retrieved and stored in a list locally accessible by the inventory system;
determine if the prescription drug claim is eligible to be fulfilled under the select program based at least in part upon the inclusion of the particular prescription drug in the select program according to the information retrieved from the first database over the communications network at the time of processing the prescription drug claim and a value of a first inventory level for the first inventory associated with the select program;
approve the prescription drug claim under the select program, in response to determining dispensing the prescription drug from the first inventory is (1) more advantageous to a patient requesting the prescription drug and (2) a value of the inventory level for the prescription drug as associated with the first inventory is above a threshold value; and
transmit notification of the claim approval and reduce the value of the inventory level associated with the first inventory for the select program by an amount of the prescription drug claim, in response to transmitting the notification of the claim approval.

2. A non-transitory computer-readable storage medium storing instructions that, when executed by a processor, cause the processor to perform operations comprising:
receiving, over a computer network, information indicative of a prescription drug claim for a prescription drug associated with a patient;
determining, from a remote database communicatively coupled to the computer network, whether the prescription drug is included in a select program at the time of processing the prescription drug claim;
determining, based on an electronic inventory system communicatively coupled to the remote database and in response to determining the prescription drug is included in the select program, whether the prescription drug claim is eligible to be fulfilled under the select program based at least in part upon a value of an inventory level associated with the select program at the time of processing the prescription drug claim, the inventory level managed by the electronic inventory system communicatively coupled to the remote database, and the inventory level associated with the select program being separate from an inventory level not associated with the select program;
determining, based on the electronic inventory system communicatively coupled to the remote database, the value of the inventory level associated with the select program is above a threshold value;
in response to determining the value of the inventory level associated with the select program is above the threshold value, approving the claim under the select program; and
providing, over the computer network, an instruction to the electronic inventory system to synchronize the value of the inventory level at the remote database associated with the select program by a quantity of the prescription drug claim claimed by the patient,
wherein the select program is a discount program.

3. An electronic inventory system communicatively coupled to one or more databases and client devices in an electronic network for updating inventory data in at least one of the databases, the electronic inventory system comprising at least one processor configured to perform operations comprising:
determining whether a prescription drug included in a select program, according to data stored in a database remotely coupled to the electronic inventory system, is eligible to be filled under the select program based at least in part on a first inventory level value associated with a first inventory maintained separately for the select program at the time of processing a prescription drug claim, the first inventory managed by the electronic inventory system communicatively coupled to the remote database, and the first inventory being separate from a second inventory other than the first inventory, the second inventory being associated with prescription drugs not qualified under the select program;
determining, based on the electronic inventory system, the first inventory level value associated with the select program is above a threshold value;
approving the prescription drug claim under the select program; and
updating, based on a quantity of the prescription drug approved, the first inventory level value.
